(19) United States
(12) Patent Application Publication
(10) Pub. No.: US 2004/0210273 A1
(43) Pub. Date: Oct. 21, 2004

(54) Presentation of patient image with implantable medical device information

(75) Inventor: Li Wang, White Bear Township, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/418,855

(22) Filed: Apr. 18, 2003

(51) Int. Cl. A61N 1/362

(52) U.S. Cl. 607/59; 607/60; 607/31

(57) ABSTRACT

On occasion, a physician may wish to monitor, test, adjust, or otherwise interact with an implantable medical device (IMD) implanted in the body of a patient, but the patient is not physically present. The physician may receive information from the IMD remotely. To better prompt the physician to recall the patient, the invention provides techniques for presenting the image of the patient, along with information pertaining to the IMD implanted in the patient. In one embodiment, the invention is directed to a method that includes storing a digital image of a patient in memory in the IMD. In other embodiments the invention is directed to methods for communicating information so that an image of the patient may be presented along with information pertaining to the IMD.

### TECHNICAL FIELD

The invention relates to medical devices, and in particular, to implantable medical devices that interact with external devices.

### BACKGROUND

When a patient having an implantable medical device (IMD) visits his personal physician, the physician may be reminded of the patient by the patient's appearance. It is one thing to see text giving the name of the patient, the history of the patient and information about the implanted device or devices, and another thing to see the patient's face. The visual appearance of the patient's face can prompt the personal physician to recall the patient and the patient's unique circumstances more easily. As a result, the physician is better enabled to focus in on the needs and problems of the particular patient.

### SUMMARY

There are many occasions in which the personal physician desires to monitor, test, adjust, or otherwise interact with the IMD, but the patient is not present. For example, the physician may wish to monitor the operation of the IMD while the patient is at home. In another example, the patient may be traveling, and may be far from the physician's office when a problem arises with the IMD. The physician may wish to interact remotely with the IMD. In scenarios such as these, the patient is not present, so the appearance of the patient cannot prompt the physician to recall the patient.

The invention is directed to techniques that present the image of the patient to a person such as the personal physician for patient, along with information pertaining to the IMD implanted in the patient, such as operating parameters. Operating parameters represent any information pertaining to programming or operation of the IMD, and depend upon the kind of IMD implanted. An operating parameter for a pacemaker, for example, may include a pacing mode, and an operating parameter for an implanted drug pump may include a drug dosage rate. Operating parameters may also include information pertaining to the condition of the patient, such as a diagnosis or a record of response to therapy. In addition to the image of the patient and operating parameters, the information presented may include identification data that identifies the implanted device, personal information about the patient, contact information concerning the personal physician, and the like. When the physician sees the textual information about the patient, along with a digital image of the patient, the physician is prompted to recall the patient.

In one embodiment, the invention is directed to a method comprising storing a digital image of a patient in memory in a medical device implanted in the body of the patient. The method may further include transmitting the digital image to an external device and may also comprise transmitting device identification data and operating parameters to the external device.

In another embodiment, the invention is directed to a method comprising transmitting an operating parameter for an implantable medical device for a patient to a remote location, and transmitting a digital image of the patient to the remote location. In an additional embodiment, the invention presents a method comprising receiving an operating parameter for an implantable medical device for a patient from a first remote location, receiving a digital image of the patient from a second remote location, and displaying the digital image and the operating parameter. The method may include receiving the operating parameter and digital image from the same location or from different locations.

In further embodiments, the invention is directed to a computer-readable medium containing instructions that cause a programmable processor to carry out one or more of the methods described above.

In an another embodiment, the invention presents a system comprising a first external device and a second external device. The first external device receives an operating parameter from an implantable medical device for a patient. The second external device receives the operating parameter from the first external device and also displays a digital image of the patient and the operating parameter.

The details of one or more embodiments of the invention are set forth in the accompanying drawings and the description below. Other features and inventive aspects of the invention will be apparent from the description and drawings, and from the claims.

### BRIEF DESCRIPTION OF DRAWINGS

FIG. 1 is a perspective diagram showing a programmer displaying a digital image of a patient with other information.

FIG. 2 is a screen shot showing the screen of the programmer depicted in FIG. 1, illustrating the digital image of the patient, device identification data, operating parameters, and other information.

FIG. 3 is a system diagram showing an exemplary system that illustrates the techniques of the invention and scenarios in which the techniques may be applied.

FIG. 4 is a system diagram showing another exemplary system that illustrates the techniques of the invention and scenarios in which the techniques may be applied.

FIG. 5 is a flow diagram illustrating an interaction between an implantable medical device and at least one external device.

FIG. 6 is a flow diagram illustrating another interaction between an implantable medical device and at least one external device.

### DETAILED DESCRIPTION

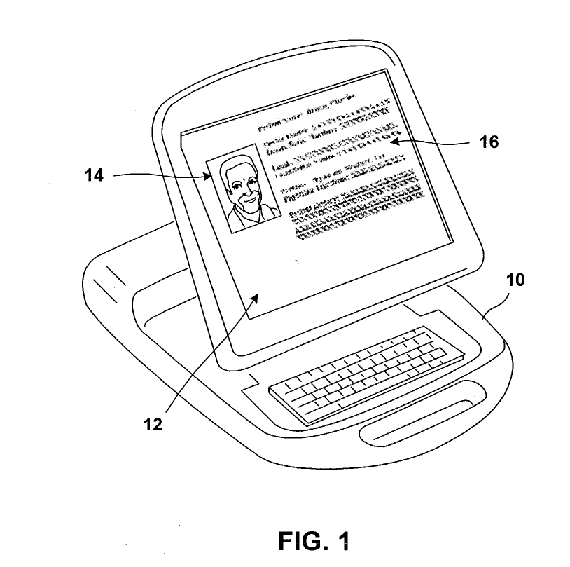

FIG. 1 is a perspective diagram of an external device 10 that includes a display 12 for presenting information about a patient. As will be described below, external device 10 may communicate with an implantable medical device (IMD) implanted in the body of the patient. External device 10 may also communicate with another external device that can communicate with the IMD. The patient and the IMD may be remote from external device 10.

In a typical embodiment of the invention, external device 10 comprises a programmer that communicates wirelessly with an IMD. The programmer may include a device such as an antenna (not shown) that facilitates wireless communication with the IMD. The programmer may transmit information to the IMD, including programming instructions and data such as digital image data. The IMD includes memory to store programming instructions and other information received from external device 10. The programmer may also receive information from the IMD.

Display 12 of external device 10 may include a cathode ray tube, liquid crystal display, light emitting diode display, plasma display or the like. Display 12 presents information to an operator, including a digital image 14 of the patient. In addition, display 12 presents information in the form of text 16 as well.

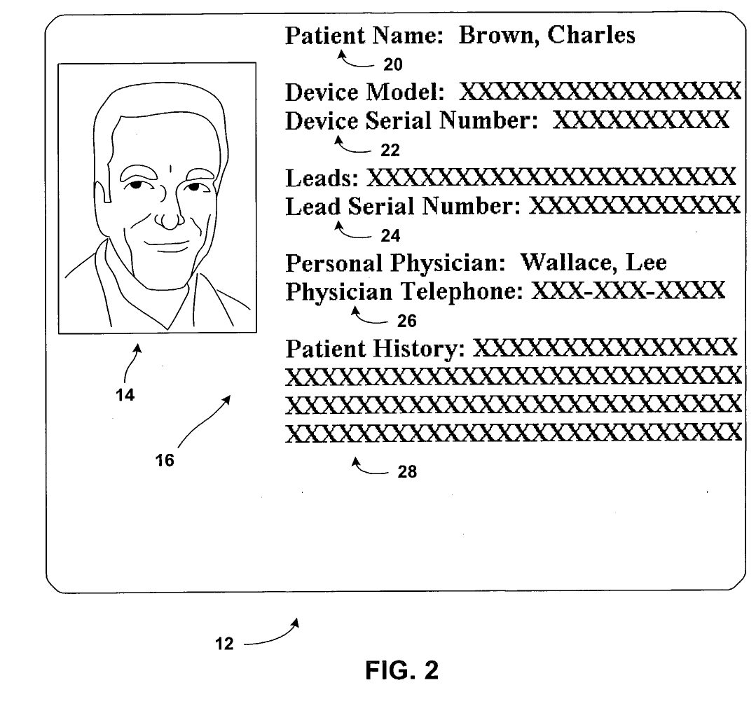

FIG. 2 is a screen shot showing information displayed on an exemplary display 12. The information includes a digital image 14 of the patient, with text 16 pertaining to the patient. Text 16 includes the patient name 20. Text 16 further includes device identification data, such as information about the specific IMD 22 and leads 24 implanted in the patient. Text also includes additional information pertaining to the patient and the implanted devices, such as the name and telephone number of the physician 26 caring for the patient, as well as patient medical history 28. Patient medical history 28 includes any other pertinent information, such as device implantation date, therapies administered by the IMD and operating parameters.

Operating parameters represent any information pertaining to the condition of the IMD and any information pertaining to the condition of the patient. Operating parameters encompass information pertaining to the programming, operation or status of the IMD, such as available battery power, as well as information pertaining to particular functions of the IMD. When the IMD is a pacemaker, for example, the operating parameters may include information such as the pacing mode and the activated antitachycardia pacing functions. When the IMD is an implanted drug pump, the operating parameters may include information such as the drug or drugs administered and the dosage rates. Operating parameters also encompass information pertaining to the condition of the patient, such as a diagnosis of the patient, a record of episodes of tachycardia, or a response to administration of a drug dosage.

Text 16 includes information that is useful to a clinician who is seeing the patient, such as the patient's implanting physician or the patient's follow-up physician. Digital image 14 is associated with text 16, thereby informing the clinician that the textual information 16 is applicable to a patient having a particular appearance. When the patient makes an in-person visit to the clinician, and the clinician uses external device 10 to communicate with an IMD implanted in a patient, the clinician may confirm that the displayed data is indeed data applicable to that patient by comparing the appearance of the patient with digital image 14.

Although the invention is useful during such in-person visits, the invention is also useful when the patient is remote from the clinician. The patient may be located in a nearby room, for example, or may be in another building, or another city, or another country. When the patient is remote, the clinician may apply the invention to confirm the identity of the patient.

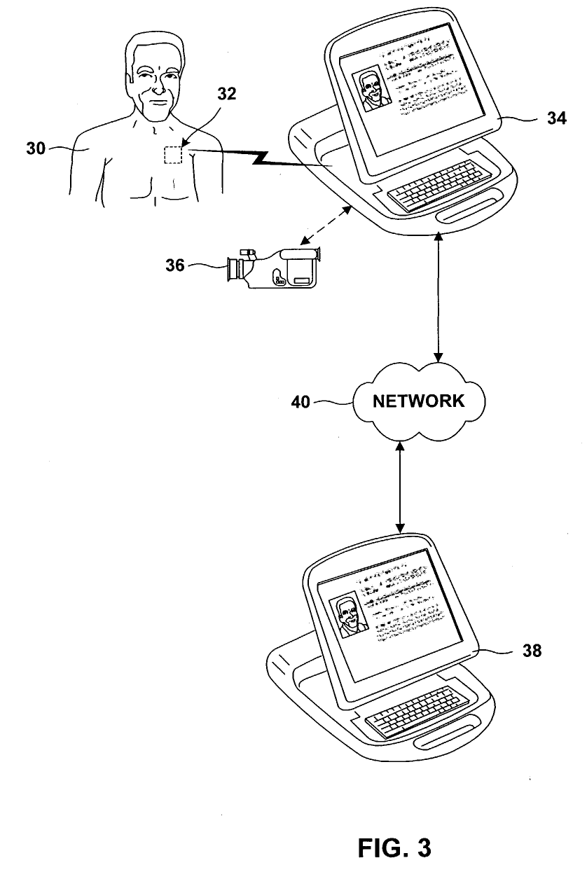

FIG. 3 is a system diagram illustrating scenarios in which the invention may be applied. In the example of FIG. 3, patient 30 has an IMD 32 implanted in his body. IMD 32 may comprise a pacemaker, pacemaker/cardioverter/defibrillator, drug pump, heart monitor, blood pressure monitor, and any other implantable medical device, along with associated hardware such as leads, catheters and the like. IMD 10 communicates wirelessly with a first external device, such as a programmer 34.

In one scenario, a camera 36 is coupled to programmer 34. In this scenario, patient 30 is in the office of a clinician, such as the personal physician for patient 30. Programmer 34 and camera 36 are also in the office of the clinician. Camera 36 captures a digital image of patient 30 and supplies the image to programmer 34. The digital image may be in any desired format, such as a bitmap or a JPEG format. The digital image comprises a still picture or a moving picture. Programmer 34 transmits the digital image to IMD 32, which stores the digital image in the memory of IMD 32. As will be described in other scenarios discussed below, the digital image stored in IMD 32 may be retrieved from IMD 32 at a later time for patient identification.

Image capture and image storage need not occur during the same office visit. In other words, a digital image of patient 30 obtained on one occasion need not be stored in IMD 32 at the same time. The clinician may, for example, scan a photograph of patient 30 at one time and download that photograph to IMD 32 for storage at a later time.

In a second scenario, patient 30 is in the office of a clinician, but the clinician is not the personal physician of patient 30. This scenario may occur, for example, if the patient experiences problems with IMD 32 while traveling. Patient 30, made aware of the problems, consults a local clinician. The local clinician in turn may wish to consult the personal physician for patient 30.

Programmer 34, in possession of the local clinician, communicates wirelessly with IMD 32. Programmer 34 may include, but is not required to include, a device that facilitates wireless communication with IMD 32. One such device is an antenna (not shown), which facilitates wireless communication when the antenna is held in proximity to IMD 32. Programmer 34 receives from IMD 32 information pertaining to patient 30 and the implanted device or devices. The received information includes, for example, the name of patient 30, device identification data, the name and telephone number of the personal physician for patient 30, operating parameters and other data. In addition, programmer 34 receives the digital image of patient 30 stored in the memory of IMD 32.

The local clinician wishes to consult with the personal physician for patient 30, who is at a site remote from patient 30. The personal physician for patient 30 has access to a second device such as a programmer 38. Local programmer 34 is in communication with remote programmer 38 via communication network 40. Network 40 may include any communication network, including a telephone network or the Internet.

Information communicated from local programmer 34 to remote programmer 38 includes information received from IMD 32, including device identification data and operating parameters. In addition, programmer 38 receives the digital image of patient 30. Even though the patient is not present in the office of the personal physician, the personal physician sees the digital image of patient 30. The personal physician can use this digital image to recall patient 30, e.g., to confirm the identity of patient 30 or to be reminded of the details of the case of this particular patient 30.

In other words, it is helpful for physicians to see text data about their patients, along with device identification data and operating parameters. Such textual data, by itself, might not prompt the physician to recall a particular patient, however, especially when the patient is not present in the office. It is believed that the personal physician is more likely to recall the patient and the patient's unique circumstances when the personal physician is presented with an image of the patient. With the personal physician prompted as to the identity of the patient, and with the personal physician receiving information such as device identification data and operating parameters associated with that patient, the personal physician is in a better position to understand the importance of the information. The personal physician is also in a better position to consult with the local clinician.

The advantages of prompting the personal physician as to the identity of the patient can extend beyond consultation. In some systems, the personal physician can interact remotely with IMD 32 via programmers 34 and 38, can troubleshoot IMD 32, and can program IMD 32 with new or additional instructions. By seeing the image of patient 30, the personal physician is in a better position to troubleshoot potential problems and program IMD 32 appropriately.

In a third scenario, the local clinician seeing patient 30 uses programmer 34 to receive from IMD 32 information pertaining to patient 30 and the implanted device or devices, such as operating parameters and other data. The local clinician once again wishes to consult the personal physician for patient 30.

A useful digital image of patient 30, however, is unavailable. It is possible, for example that no digital image was ever stored in IMD 32. It is also possible that a digital image was stored in IMD 32, but the digital image does not provide an accurate picture of the patient, due to corruption of the image, a change in the appearance of the patient, or exhibition of visual symptoms that are not present in the digital image.

Accordingly, the local clinician may couple camera 36 to programmer 34, and may capture a still or moving digital image of patient 30. Programmer 34 transmits this digital image, along with device identification data, operating parameters and other information received from IMD 32, to programmer 38 via network 40. The personal physician, by observation of the image data displayed on programmer 38, may be prompted to recall patient 30 because of the digital image. The digital image may also be used to diagnose the condition of patient 30 or to prescribe treatment.

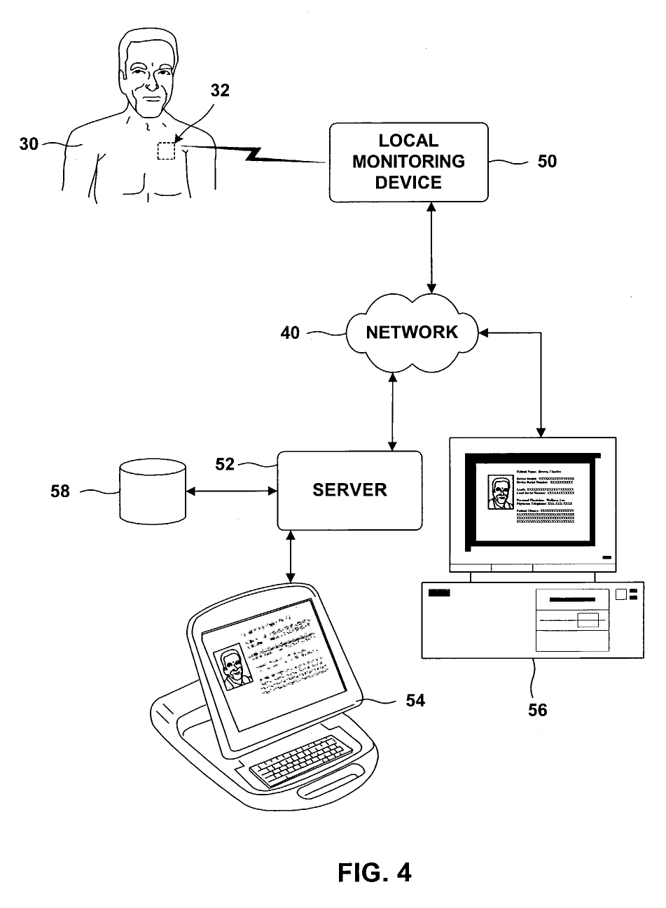

FIG. 4 is a system diagram illustrating additional scenarios in which the invention may be applied. Patient 30 is at a location away from the office of his personal physician, such as the home of patient 30. A local monitoring device 50 is an external device proximate to patient 30. Local monitoring device 50 automatically downloads information from IMD 32, including, for example, device identification data and operating parameters. In addition, local monitoring device 50 receives the digital image of patient 30 stored in the memory of IMD 32. Local monitoring device 50 sends this information via communication network 40 to a server 52.

Local monitoring device 50 may include, for example, an antenna that facilitates communication with IMD 32 when the antenna is held in proximity to IMD 32. Local monitoring device 50 may also include an interface to a local communication system, such as a connection to a local telephone system. The invention is not limited to monitoring devices having such features, however.

An external device such as a programmer 54 coupled to server 52 may display the device identification data, operating parameters and digital image of patient 30. In a variation, a workstation 56 may receive the same information from server 52 via network 40. Server 52 may include security precautions to prevent unauthorized access to the information.

The personal physician, upon seeing the digital image of patient 30, may be prompted to recall the details of the case of this particular patient 30. As a result, the personal physician is more likely to recall circumstances when the personal physician is presented with an image of the patient. The personal physician is also more likely to appreciate the importance of the textual information, and may more readily evaluate whether the displayed information is of concern or not.

In a further scenario, a useful digital image of patient 30 is unavailable. Server 52 may receive data from IMD 32, such as a device serial number, and retrieve a digital image of patient 30 associated with that data from a storage device 58, which may be either local or remote. Server 52 may supply the digital image to programmer 54 or workstation 56, along with device identification data and operating parameters received from IMD 32. Programmer 54 or workstation 56 may display the digital image of patient 30 along with the device identification data or operating parameters.

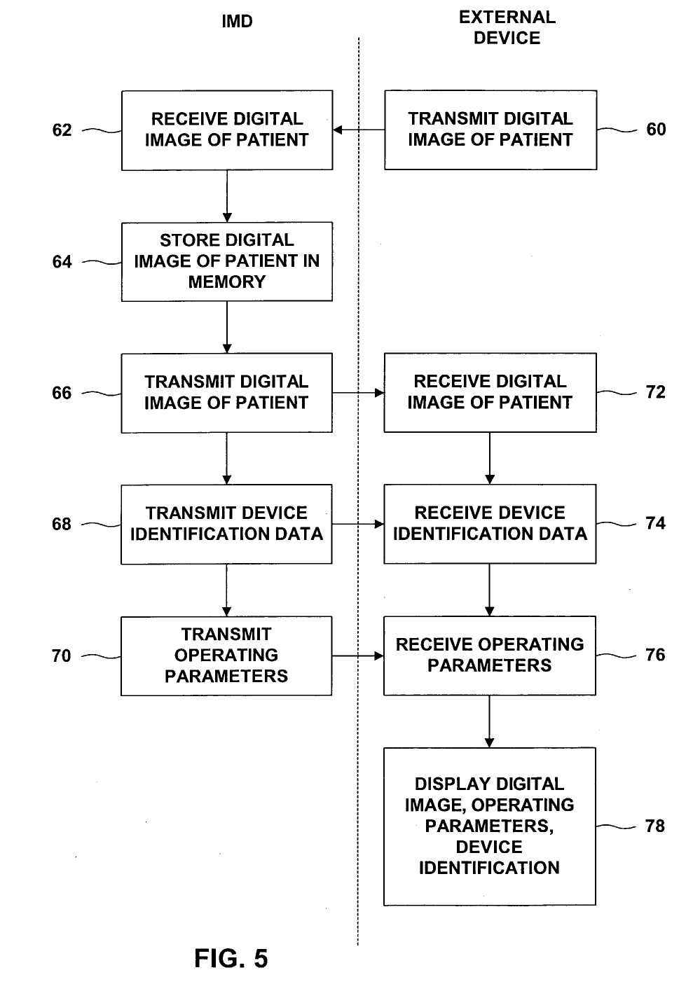

FIG. 5 is a flow diagram illustrating some of the techniques described above. An external device transmits a digital image of a patient to an IMD implanted in the body of the patient (60), which receives the image (62) and stores the image in memory (64). At a later time, in response to an interrogation from another device or as part of a scheduled communication to an external device, the IMD transmits the digital image to the external device (66), along with device identification data (68) and operating parameters (70). The operating parameters may include any information pertaining to the operation or status of the IMD, and may also include any information pertaining to a diagnosis of the patient or any other information bearing upon the condition of the patient. An external device, not necessarily the same external device that transmitted the digital image (60), receives the information (72, 74, 76). The external device may receive the information (72, 74, 76) directly, or via another device and a communication network.

The external device displays information received from the IMD (78). In particular, the external device displays the digital image and pertinent information applicable to the patient shown in the digital image. Typically, the pertinent information includes the operating parameters of the IMD, and may also include device identification data and other data. In a variation, the external device that receives information from the IMD (72, 74, 76) is not necessarily the same external device that displays the information (78). One external device receiving the information may communicate the information to a another external device for display via a communication path such as a communication network.

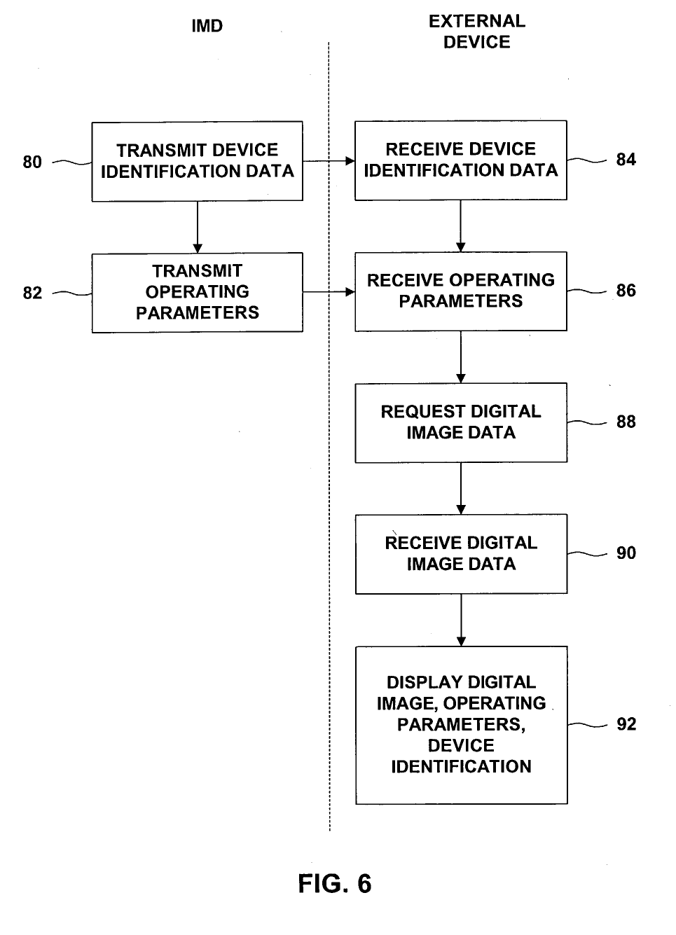

FIG. 6 is a flow diagram illustrating additional techniques described above. In response to an interrogation from another device or as part of a scheduled communication to an external device, or for another reason, the IMD transmits device identification data to the external device (80) and operating parameters (82). The external device receives the information (84, 86).

The external device does not receive a useful digital image of the patient from the IMD. Instead, the external device receives the digital image data from another source. The external device can, for example, receive the image data from a camera at the location of the patient. In another implementation, the external device makes a request for the digital image data (88) from a local or remote storage device, and receives the image data (90) in response to the request. The external device displays the digital image and pertinent information, such as the operating parameters of the IMD (92).

Many embodiments of the invention have been described. Various modifications may be made without departing from the scope of the claims. For example, the invention is not limited to application with the particular implantable medical devices described above, but may be practiced by a wide variety of implantable medical devices. Similarly, the invention is not limited to application with the particular external devices described above, but may be implemented with other external devices, such as a personal digital assistant or a cellular telephone.

Nor is the invention limited in application to the particular illustrative scenarios provided above. The invention may be useful in a variety of circumstances, and may be especially helpful to the physician any time the physician desires to review information about the patient when the patient is not present. The invention may also be used by persons other than physicians.

In addition, the invention may be embodied as a computer-readable medium that includes instructions for causing a programmable processor to carry out the methods described above. A “computer-readable medium” includes but is not limited to read-only memory, Flash memory and a magnetic or optical storage medium. The instructions may be implemented as one or more software modules, which may be executed by themselves or in combination with other software.

These and other embodiments are within the scope of the following claims.

1. A method comprising storing a digital image of a patient in memory in a medical device implanted in the body of the patient.

2. The method of claim 1, further comprising transmitting the digital image from the medical device to an external device.

3. The method of claim 2, further comprising transmitting device identification data to the external device.

4. The method of claim 2, further comprising transmitting operating parameters to the external device.

5. The method of claim 2, wherein the external device comprises a programmer that communicates with the medical device.

6. The method of claim 1, wherein the medical device comprises a pacemaker.

7. The method of claim 1, further comprising receiving the digital image.

8. A method comprising:
transmitting an operating parameter for an implantable medical device for a patient to a remote location; and
transmitting a digital image of the patient to the remote location.

9. The method of claim 8, wherein the implantable medical device comprises a pacemaker.

10. The method of claim 8, further comprising receiving the operating parameter from the implantable medical device.

11. The method of claim 8, further comprising receiving the digital image of the patient from the implantable medical device.

12. The method of claim 8, wherein the operating parameter comprises at least one of information pertaining to a condition of the implantable medical device and information pertaining to a condition of the patient.

13. A method comprising:
receiving an operating parameter for an implantable medical device for a patient from a first remote location;
receiving a digital image of the patient from a second remote location; and
displaying the digital image and the operating parameter.

14. The method of claim 13, wherein the implantable medical device comprises a pacemaker.

15. The method of claim 13, wherein the first and second locations are the same location.

16. The method of claim 13, further comprising transmitting programming instructions to the implantable medical device.

17. The method of claim 13, wherein the operating parameter comprises at least one of information pertaining to a condition of the implantable medical device and information pertaining to a condition of the patient.

18. A system comprising:
a first external device to receive an operating parameter from an implantable medical device for a patient; and
a second external device to receive the operating parameter from the first external device and to display a digital image of the patient and the operating parameter.

19. The system of claim 18, further comprising a communication network, wherein the second external device receives the operating parameter from the first external device via the communication network.

20. The system of claim 18, further comprising a camera coupled to the first external device to capture the digital image of the patient.

21. The system of claim 18, further comprising a storage device to store the digital image of the patient, wherein the second external device is further configured to receive the digital image of the patient from the storage device.

22. The system of claim 18, wherein the operating parameter comprises at least one of information pertaining to a condition of the implantable medical device and information pertaining to a condition of the patient.

23. A computer-readable medium comprising instructions for causing a programmable processor to store a digital image of a patient in memory in a medical device implanted in the body of the patient.

24. The medium of claim 23, the instructions further causing the processor to transmit the digital image to an external device.

25. The medium of claim 24, the instructions further causing the processor to transmit device identification data to the external device.

26. The medium of claim 24, the instructions further causing the processor to transmit operating parameters to the external device.

27. The medium of claim 23, the instructions further causing the processor to receive the digital image.

28. A computer-readable medium comprising instructions for causing a programmable processor to:
transmit an operating parameter for an implantable medical device for a patient to a remote location; and
transmit a digital image of the patient to the remote location.

29. The medium of claim 28, the instructions further causing the processor to receive the operating parameter from the implantable medical device.

30. The medium of claim 28, the instructions further causing the processor to receive the digital image of the patient from the implantable medical device.

31. The medium of claim 28, wherein the operating parameter comprises at least one of information pertaining to a condition of the implantable medical device and information pertaining to a condition of the patient.

32. A computer-readable medium comprising instructions for causing a programmable processor to:
receive an operating parameter for an implantable medical device for a patient from a first remote location;
receive a digital image of the patient from a second remote location; and
display the digital image and the operating parameter.

33. The medium of claim 32, wherein the first and second locations are the same location.

34. The medium of claim 32, the instructions further causing the processor to transmit programming instructions to the implantable medical device.

35. The medium of claim 32, wherein the operating parameter comprises at least one of information pertaining to a condition of the implantable medical device and information pertaining to a condition of the patient.
